(19) United States
(12) Patent Application Publication
(10) Pub. No.: US 2011/0224541 A1
(43) Pub. Date: Sep. 15, 2011

(54) METHODS AND ARRANGEMENTS FOR ANALYSIS, DIAGNOSIS, AND TREATMENT MONITORING OF VOCAL FOLDS BY OPTICAL COHERENCE TOMOGRAPHY

(75) Inventors: Seok-Hyun Yun, Cambridge, MA (US); James B. Kobler, Andover, MA (US)

(73) Assignee: The General Hospital Corporation, Boston, MA (US)

(21) Appl. No.: 12/963,496

(22) Filed: Dec. 8, 2010

Related U.S. Application Data
(60) Provisional application No. 61/267,780, filed on Dec. 8, 2009.

(51) Int. Cl. A61B 6/00

(52) U.S. Cl. 600/425

(57) ABSTRACT

Exemplary embodiments of an apparatus and a method can be provided. For example, a first information can be obtained for at least one signal that is (i) at least partially periodic and (ii) associated with at least one structure. In addition, a second information associated with the structure can be generated at multiple time points within a single cycle of the at least one signal. The second information can include information for the structure below a surface thereof. Further, it is possible to generate a third information based on the first information and the second information, where the third information is associated with at least one characteristic of the structure.

### CROSS-REFERENCE TO RELATED APPLICATION(S)

This application is based upon and claims the benefit of priority from U.S. patent application Ser. No. 61/267,780, filed on Dec. 8, 2009, the entire disclosure of which is incorporated herein by reference.

### FIELD OF THE DISCLOSURE

Exemplary embodiments of the present disclosure relate to the utilization of optical coherence tomography for obtaining information regarding at least one anatomical structure, and more particularly to exemplary methods and arrangements for analysis, diagnosis, and treatment monitoring of vocal folds using optical coherence tomography procedures.

### BACKGROUND INFORMATION

Voice disorders can disrupt normal human communication causing far-reaching negative personal and social-economic consequences for those affected. It is estimated that about 7.5 million Americans suffer from voice disorders. One of the main causes of voice disorders can be damage to the subepithelial layers of laryngeal vocal fold tissue that must vibrate periodically and at high frequencies (e.g., 100-1,000 Hz) to produce a normal voice.

The paired vocal folds, located inside the larynx (as shown in FIG. 1), provide an interesting and highly efficient biomechanical system for a sound generation. To generate voice sounds, the vocal folds are first abducted for inspiration (as shown in a left portion of FIG. 1), and then adducted (as shown in a right portion of FIG. 1) during exhalation. As air flows past, aerodynamic forces and the intrinsic elasticity of the vocal fold tissue set the folds into periodic oscillation. The air steam is thereby modulated, generating an acoustic buzz we hear as the voice. At low vocal frequencies (e.g., at about 100 Hz in males, and at about 200 Hz in females), waves (e.g., mucosal waves) that are about 1-2 mm in amplitude ripple across the vocal folds from inferior to superior with each cycle of vibration. At higher frequencies, the mucosal waves can become more rapid and shallow. Detailed biomechanics and aerodynamics underlying voice production may still not be completely understood, although the periodic and symmetrical motions of the mucosal waves to valve the airflow can be important. Thus, diseases or injuries that affect these waves can often result in voice disorders.

The mucosal waves can be made possible by the presence of a layer of extremely soft and elastic connective tissue just beneath the epithelium, called the superficial lamina propria (“SLP”). The SLP is about 1 mm thick and is rich in hyaluronic acid, a resilient extracellular matrix molecule that is also abundant in the vitreous humor of the eye and nucleus pulposus of the intervertebral disks. A healthy layer of SLP is important to a good voice, but the SLP in a vulnerable location, and is frequently damaged by diseases or trauma. Other diseases that thicken and stiffen the epithelium, such as cancer and papilloma can also have significant impacts on the voice. Thus, much of the essential dynamics in voice production and most laryngeal disease are localized to the superficial 1-2 mm of the vocal fold tissue that includes the epithelium and the SLP. One problem in the field of Laryngology is how to best treat diseases that affect these thin layers while preserving the mucosal wave and good voice production.

To evaluate the health of the vocal folds, laryngologists and speech language pathologists generally rely on laryngeal videostroboscopy. Videostroboscopy (as described in less, D. M., Hirano, M. & Feder, R. J., Videostroboscopic Evaluation of the Larynx, Ear Nose & Throat Journal vol. 66, 1987) uses voice-triggered stroboscopic illumination in combination with transoral or transnasal endoscopes, for observing and recording vocal fold motion (see FIG. 1). Despite the ubiquity and utility of videostroboscopy, this procedure is highly qualitative, and the data obtained can be quite subjective. Therefore, an analysis of vocal fold vibration can be greatly improved if a procedure becomes available for capturing the three-dimensional (3D) motions of the vocal folds quantitatively and with high temporal and spatial resolution. Such a method could reduce subjectivity and make laryngeal exams more reliable and amenable to biomechanical analysis, rather than relying on visual impressions. Parameters such as amplitude, symmetry, velocity and wavelength of mucosal waves could be compared before and after treatment or between normal and diseased vocal folds. High-speed imaging overcomes some of the limitations of stroboscopy; however, it is still a 2D method limited to viewing the vocal fold surfaces. (See Kendall, K. A., High-Speed Laryngeal Imaging Compared With Videostroboscopy in Healthy Subjects, Archives of Otolaryngology-Head & Neck Surgery vol. 135, pp. 274-281, 2009).

Dynamic cross-sectional imaging can provide additional information into the anatomical and biomechanical bases of voice disorders. In addition, the ability to observe cross-sectional dynamics would permit analysis of the deformation of implanted materials designed to match the viscoelastic properties of the normal SLP. Previously, satisfactory method or system for assessing the biomechanics of these materials in situ may be unknown

Alternative approaches for capturing dynamics and/or depth information, such as ultrasound or MRI (as described in Tsai, C. G., Shau, Y. W., Liu, H. M. & Hsiao, T. Y., Laryngeal mechanisms during human 4-kHz vocalization studied with CT, videostroboscopy, and color Doppler imaging, Journal of Voice 22, 275-282, 2008, and Ahmad, M., Dargaud, J., Morin, A. and Cotton, F. Dynamic MRI of Larynx and Vocal Fold Vibrations in Normal Phonation. Journal of Voice, vol. 23, pp. 235-239, 2009) may not be satisfactory due to suboptimal temporal and/or spatial resolution.

Optical coherence tomography (OCT) is an optical procedure that can utilize interferometry of backscattered near-infrared light to image cross-sections of tissue in patients, with a resolution of typically about 10 μm. Time-domain OCT has become an important diagnostic imaging tool in ophthalmology. (See Huang, D. et al., Optical coherence tomography, Science 254, pp. 1178-81 (1991)). OCT has also shown promise in identifying dysplasia in Barrett's esophagus and colonic adenomas, for discerning all of the histopathologic features of vulnerable coronary plaques, and for static imaging of vocal fold mucosa and vocal fold pathology. (See Burns, J. A. et al., Imaging the mucosa of the human vocal fold with optical coherence tomography, Annals of Otology Rhinology and Laryngology 114, 671-676 (2005); Vokes, D. E. et al., Optical coherence tomography-enhanced microlaryngoscopy: Preliminary report of a noncontact optical coherence tomography system integrated with a surgical microscope, Annals of Otology Rhinology and Laryngology 117, pp. 538-547 (2008); Kraft, M. et al., Clinical Value of Optical Coherence Tomography in Laryngology, Head and Neck-Journal for the Sciences and Specialties of the Head and Neck 30, pp. 1628-1635 (2008); and Boudoux, C. et al., Optical Microscopy of the Pediatric Vocal Fold, Archives of Otolaryngology-Head & Neck Surgery 135, pp. 53-64 (2009)).

However, until recently, OCT procedure has been too slow for providing a comprehensive 3D microscopic imaging, and therefore has been relegated to a point-sampling technique with a field of view comparable to a conventional biopsy. The application of Fourier-domain ranging techniques, instead of the delay-scanning interferometry of OCT, has led to an improvement in a detection sensitivity. Such procedure, i.e., optical frequency domain interferometry (OFDI) leverages high sensitivity to provide orders of magnitude faster imaging speed compared to the conventional OCT procedure.

The image acquisition speed provided by the OFDI techniques, however, may not be fast enough to capture vocal fold motion directly. One exemplary OFDI system can acquire about 50,000 (continuous) to 370,000 (short burst) axial line (A-line) scans per second. For example, to obtain a single image frame containing about 1,000 A-lines, the OFDI system can takes about 3-20 ms. This frame acquisition time can be too slow to image the vocal folds, which vibrate at frequencies of about 100-1000 Hz. To capture such a fast motion directly, without motion artifacts, the frame rate would have to be much higher than 10 kHz (10-100 phases), which would likely use an A-line rate to be higher than 10 MHz. Such specification may not currently be attainable due to various technical problems. Furthermore, it can result in a substantially decreased signal-to-noise ratio (SNR) and clinically unacceptable poor image quality.

Thus, it may be beneficial to address and/or overcome at least some of the deficiencies of the prior approaches, procedures and/or systems that have been described herein above.

### OBJECTS AND SUMMARY OF EXEMPLARY EMBODIMENTS OF PRESENT DISCLOSURE

Exemplary embodiments of the present disclosure can address at least most of the above-described needs and/or issues by facilitating imaging of the vocal fold motion quantitatively with four-dimensional (e.g., 4D: x,y,z and time) resolution. The exemplary embodiments of the present disclosure can utilize Fourier-domain optical coherence tomography (OCT)—herein also referred to as optical frequency domain imaging (OFDI), a procedure that is described in, e.g., S. H., Tearney, G. J., de Boer, J. F., Iftimia, N. & Bouma, B. E., High-speed optical frequency-domain imaging, Optics Express 11, pp. 2953-2963 (2003). An exemplary embodiment of the procedure, system and method according to the present disclosure can facilitate a production of a sequence of high-resolution 3D images of the vocal folds over a full cycle of vibration. In combination with standard laryngeal endoscopes, such exemplary embodiments can be used in a similar way as conventional stroboscopy is used, while facilitating the examination of not only the surface, but also the motion of the entire volume of the essential superficial tissues, quantitatively.

To rapidly image vibrating vocal folds, according to one exemplary embodiment of the present disclosure, image acquisition methods can be provided which can rely on a use of a voice signal from a microphone, an electroglottograph (EGG) or a subglottic pressure transducer for synchronization. Stable phonation and repeatable triggering, as used in conventional stroboscopy, is necessary. The probe laser beam can be scanned across the vocal fold, acquiring axial profiles at each spatial location and each temporal phase of motion. A subsequent image reconstruction based on the timing synchronization with the voice signal will produce a sequence of high-resolution 3D images of the vocal folds over a full cycle of vibration. A dynamic cross-sectional imaging of vibrating vocal folds can be achieved, which has not been previously obtained demonstrated.

For example, 4D vocal fold imaging of a patient and animal models can be expected to certain exemplary impacts.

Improved diagnosis of voice disorders: The exemplary embodiments of the present disclosure can facilitate the clinicians to compare volumetric vocal fold motion of normal and diseased vocal folds quantitatively and observe the location and extent of subsurface pathology in both dynamic and static modes. This can elucidate how pathologies affect vocal fold motion and resulting voice quality, which in turn should lead to improvements in treatment methods.

Assessment of the efficacy of surgery and treatments designed to improve vocal fold function: The main cause of chronic dysphonia or voice loss is permanent damage to the normal soft tissue in superficial lamina propria due to disease or trauma. Exemplary treatment approaches include bio-implants and surgical techniques that are designed to restore the vibratory properties of damaged vocal fold phonatory mucosa. High-speed four-dimensional (4D) OFDI imaging has potential to facilitate an elastographic measurement of biomechanical properties, such as elastic modulus, of the vocal folds and of the implants, which should facilitate optimization of this treatment approach.

Indeed, exemplary embodiments of the present disclosure provide endoscopic technology methods, systems and arrangements can be provided which can facilitate with the diagnosis and treatment of patients with voice disorders. For example, it is possible to use high-speed optical coherence tomography (OCT) methods and systems, combined with physiological triggering, to image vibrating vocal folds with high spatial and temporal resolution. Oscillations of the surface and interior structure of the vocal fold can then be viewed in slow-motion, providing essentially a dynamic histological cross-section. The ability to view previously hidden events and quantitatively capture the motion in three dimensions can indicate that the exemplary embodiments of the present disclosure can be useful and sought after.

To that end, exemplary embodiments of an apparatus and a method can be provided. For example, with at least one first arrangement (or a plurality of first arrangements), a first information can be obtained for at least one signal that is (i) at least partially periodic and (ii) associated with at least one structure. In addition, with at least one second arrangement (or a plurality of second arrangements), a second information associated with the structure can be generated at multiple time points within a single cycle of the at least one signal. The second information can include information for the structure below a surface thereof. Further, with at least one third arrangement (or a plurality of third arrangements, it is possible to generate a third information based on the first information and the second information, where the third information is associated with at least one characteristic of the structure.

According to one exemplary embodiment of the present disclosure, the first information can include first data for multiple time points within one cycle of such at least partially periodic signal. The third information can include at least one image associated with the structure, which can include a three-dimensional image and/or multiple sequential images over the multiple time points.

According to another exemplary embodiment of the present disclosure, the third information can include one or more of (i) velocity information of a periodic motion of the structure during the multiple time points, (ii) mechanical properties of the structure during the multiple time points, (iii) strain information for the structure, and/or (iv) further information regarding a periodic motion of the structure during the multiple time points. The structure can be (i) at least one anatomical structure, (ii) at least one vocal cord, and/or (iii) polymers or viscoelastic materials.

According to yet another exemplary embodiment of the present disclosure, the second arrangement(s) can include an optical coherence tomography arrangement. The optical coherence arrangement can be configured to transmit a radiation the structure, and to control the radiation as a function the first information provided by the first arrangement(s). The optical coherence arrangement can be facilitated in an endoscope or a catheter. The second information can include a phase interference information associated with the structure, and the third arrangement(s) can be configured to determine at least one characteristic of a motion of the structure using the phase interference information. The characteristic(s) of the motion can comprise an amplitude property of the motion. The radiation can be controlled by controlling a propagation direction of the radiation.

According to still another exemplary embodiment of the present disclosure, the first arrangement(s) can obtain the first information during a motion of the structure. A periodicity of the motion can be in a range of approximately 10 Hz and 10 KHz. The third information can be provided for an internal portion of the structure. Further, the first arrangement(s) can include one or more of (i) a piezoelectrical transducer, (ii) an ultrasound transducer, (iii) an optical position sensor, or (iv) an imaging arrangement which indicates a motion of or within the structure.

These and other objects, features and advantages of the exemplary embodiment of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying figures showing illustrative embodiments of the present disclosure, in which:

FIG. 1 are images of vocal folds using a transoral laryngoscope and strobe illumination, with the left-side image illustrating a normal vocal folds during inspiration, and a right-side image illustrating adducted vocal folds during a vibration;

FIG. 2 is a block diagram of an exemplary embodiment of an OFDI system for dynamic vocal fold imaging according to an exemplary embodiment of the present disclosure;

FIG. 3A is a diagram associated with an exemplary triggered scan procedure for high temporal resolution image acquisition and reconstruction according to an exemplary embodiment of the present disclosure which can utilize a voice signal from a microphone or electroglottograph for time synchronization;

FIG. 3B is a diagram associated with an exemplary continuous scan for an accelerated high temporal resolution image acquisition and reconstruction according to another exemplary embodiment of the present disclosure which can utilize a voice signal from a microphone or electroglottograph for time synchronization;

FIG. 4a is an exemplary configuration illustrating a vocal fold tissue on a vibrating toothbrush head according to an exemplary embodiment of the present disclosure;

FIG. 4b are exemplary reconstruction images of instantaneous snapshots of the rapidly vibrating tissue according to an exemplary embodiment of the present disclosure, with a symbol S being systole, and a symbol D being diastole;

FIG. 5 are exemplary graphs indicating exemplary data depicting a Doppler-induced artifact based on exemplary OFDI images of a moving mirror, in accordance with exemplary embodiments of the present disclosure;

FIG. 6a is an exemplary OFDI image of the vocal fold after injecting PEG into the mucosa, so as to provide exemplary data and indicate an exemplary concept of elastography for characterizing biomechanical properties of implants in the vocal folds, in accordance with exemplary embodiments of the present disclosure;

FIG. 6b are exemplary illustrations of an expected deformation of the implant in the vibrating vocal fold so as to provide the exemplary data and indicate an exemplary concept of elastography for characterizing biomechanical properties of implants in the vocal folds, in accordance with exemplary embodiments of the present disclosure;

FIG. 7a is an illustration of an exemplary vocal fold ex-vivo testing apparatus according to an exemplary embodiment of the present disclosure using which a hemisected larynx is sealed in a chamber and warm humidified air is blown past the vocal fold, which is apposed to a glass slide;

FIG. 7b is an enlarged illustration of the bisected larynx showing vocal fold against glass; and

FIG. 8 is a block diagram of a method according to an exemplary embodiment of the present disclosure.

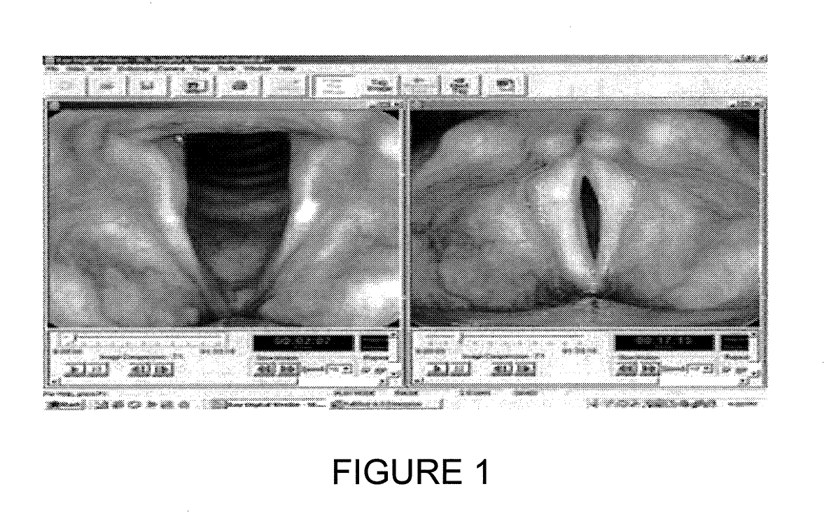

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described exemplary embodiments without departing from the true scope and spirit of the subject disclosure as defined by the appended claims.

### DETAILED DESCRIPTION OF EXEMPLARY EMBODIMENTS

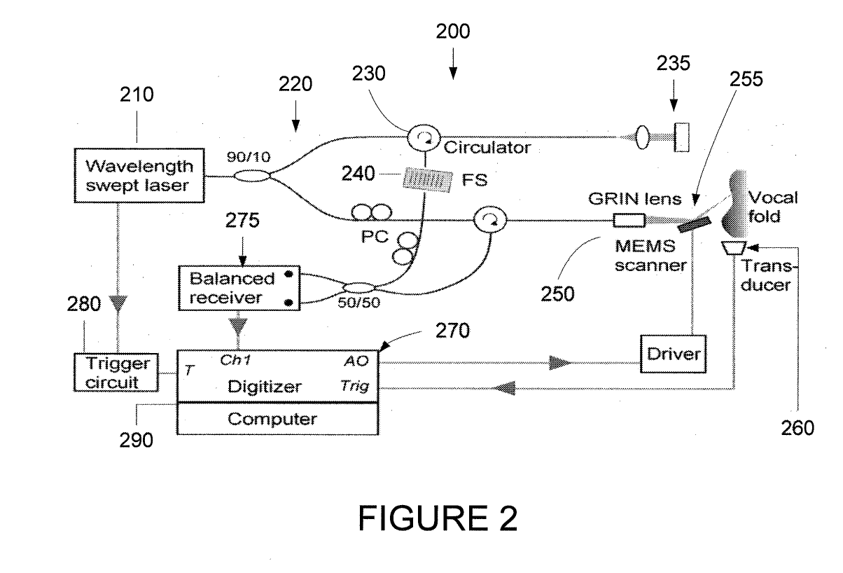

FIG. 2 shows a schematic of an exemplary embodiment of a high-speed OFDI system 200 according to an exemplary embodiment of the present disclosure. Such exemplary system 200 can utilize the following elements: a polygon-scanning semiconductor laser 210 with a sweep rate up to 100 kHz and broad tuning range at 1.3 μm; a dual-balanced polarization-diverse fiber-optic interferometer 220; a circulator 230, an acousto-optic frequency shifter 240 to receive the radiation from the circulator 230 and a reference arm 235, and to remove depth degeneracy. The exemplary system 200 also includes a probe 250 utilizing a miniature two-dimensional (2D) MEMS scanner 255, and a transducer 260 to synchronize the beam scanner 255 to the vocal fold vibration. The receiver signal can be digitized at about 50-100 MS/s by a high-speed digitizer 270 (in conjunction with the signals received from a balanced receiver 275 and a trigger circuit 280), and streamed to a hard disk for recording as well as to a computer 290 for real-time image display. It is possible to utilize such exemplary system 200 to provide certain exemplary image acquisition processing procedures as described herein.

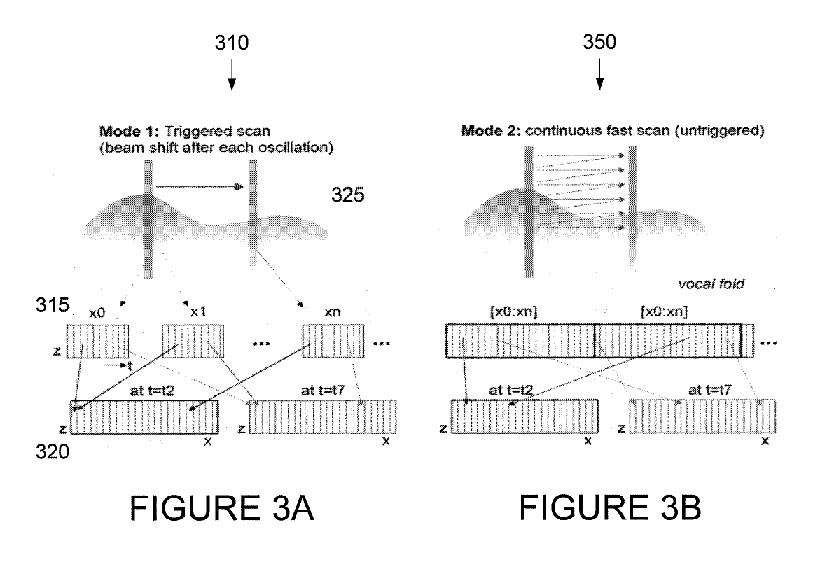

FIGS. 3A and 3B illustrates exemplary image acquisition procedures according to exemplary embodiments of the present disclosure. The acquisition modes shown in FIGS. 3A and 3B can rely on using a voice signal from a microphone or electroglottograph for synchronization. As in conventional stroboscopy, relatively stable phonation and repeatable triggering is necessary.

For example, in one exemplary high-resolution mode 310 shown in FIG. 3A (e.g., Mode-1), one vertical line 315 can be sampled repeatedly per cycle, and a positive zero-crossing of the voice waveform can trigger the beam to move to the next horizontal position 320. At each position, a series of A-lines during a single motion cycle can be recorded (M-mode). After many or all of the horizontal positions (x0, x1, . . . , xn) can be scanned, A-lines that have been captured at different positions but at the same phase of the periodic motion can be grouped together to reconstruct “snap-shot” cross-sectional images 325. These snapshots can then be rendered as frames in a video that shows high resolution motion over a complete cycle of vibration. In this exemplary mode shown in FIG. 3A, the image capture time (in seconds) can be approximately equal to the total number of acquired A-lines divided by the voice frequency. The basic principle of this exemplary technique can be referred to as a gated image acquisition that is described in Lanzer, P. et al., Cardiac Imaging Using Gated Magnetic-Resonance, Radiology 150, 121-127 (1984), and has been used with a time-domain OCT system for embryonic heart imaging at a heartbeat frequency ranging from 1 to 10 Hz. (See Jenkins, M. W., Chughtai, O. Q., Basavanhally, A. N., Watanabe, M. & Rollins, A. M., In vivo gated 4D imaging of the embryonic heart using optical coherence tomography, Journal of Biomedical Optics 12 (2007)). The implementation of an exemplary gated acquisition to the vocal fold imaging can be modified in accordance with the exemplary embodiments of the present disclosure since the vocal fold motion can be about three orders of magnitude greater (e.g., 100 times faster and 10 times larger in amplitude) than that of the embryonic heart.

FIG. 3A shown illustrations associated with another exemplary mode 350 of operation (e.g., Mode-2) that can facilitate a faster image acquisition. For example, the imaging processing arrangement can execute continuously at full speed (no triggering) and the 4D image (e.g., three spatial dimensions plus time) will be reconstructed offline by using the voice signal for timing synchronization. This exemplary mode of FIG. 3B can be advantageous for providing a global picture of vocal fold function, e.g., capturing a 3D image over the anterior-to-posterior extent of the vocal folds, including depth, over a full cycle of vibration.

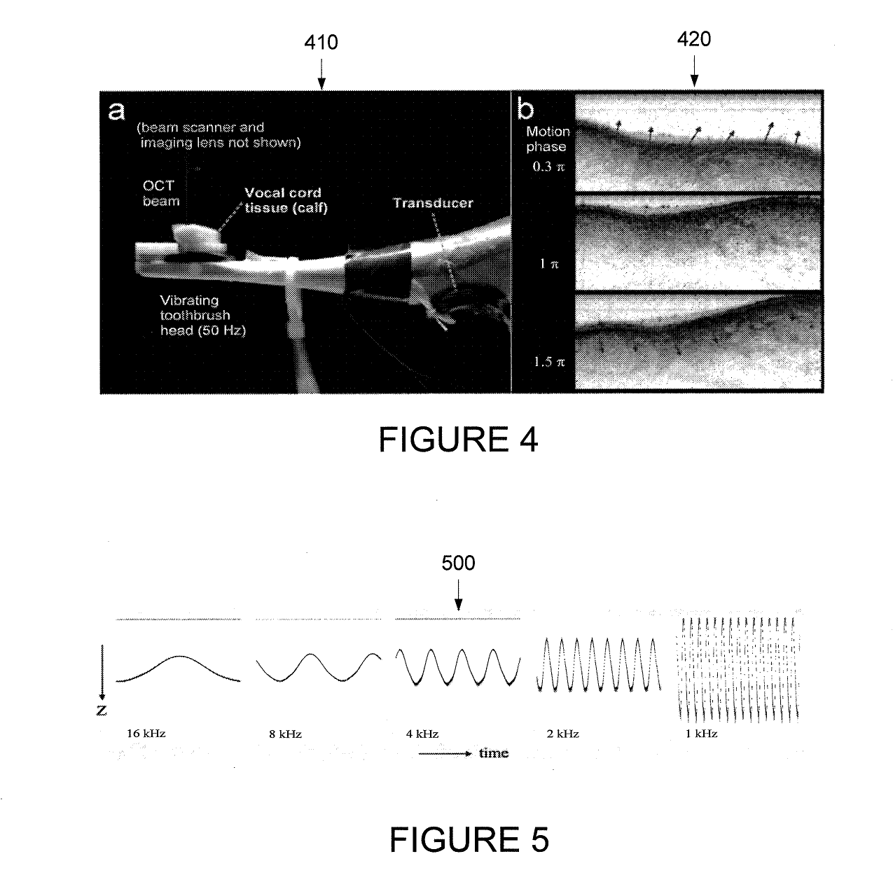

For example, Mode-1 310 of FIG. 3A can be implemented using an exemplary 10 kHz, 1.7 μm OFDI system. To simulate vocal vibration, e.g., it is possible to mount a dissected calf vocal fold on a motorized toothbrush head that oscillates sinusoidally at about 50 Hz. FIG. 4a shows an exemplary image 410 of such exemplary configuration in accordance with exemplary embodiments of the present disclosure. For example, a small magnet can be attached to the motor shaft, which provides a trigger signal through a wire pick-up coil for a time synchronization. It is possible to use a galvanometer mirror scanner, which can move the probe laser beam laterally across the tissue, e.g., in a step-wise manner upon receiving the trigger signal at approximately 50 Hz. In one example, it took 10 seconds to acquire a total of about 100,000 axial profiles at, e.g., about 500 exemplary transverse locations and 200 exemplary motion phases of vibration. Based on this exemplary data set, it is possible to reproduce, e.g., about 200 snapshot images of the cross-section of the tissue. FIG. 4b shows exemplary representative reconstructed images 420 of exemplary reconstruction of instantaneous snapshots of the rapidly vibrating tissue. Arrows in FIG. 4b indicate several exemplary local velocity vectors calculated by simple image correlation.

As with other imaging modalities, rapid large sample motion can cause various effects in the OFDI images. The theory and experimental verifications of various motion artifacts, such as SNR degradation and resolution blurring due to axial and transverse motions is described in Yun, S. H., Tearney, G. J., de Boer, J. F. & Bouma, B. E., Motion artifacts in optical coherence tomography with frequency-domain ranging, Optics Express 12, 2977-2998 (2004). One of the prominent artifacts can be the Doppler-induced distortion arising from the velocity component parallel to the optical beam axis, as shown in FIG. 5 which illustrates exemplary graphs 500 indicating exemplary data depicting a Doppler-induced artifact based on exemplary OFDI images of a moving mirror, in accordance with exemplary embodiments of the present disclosure. As indicated in FIG. 5, the exemplary OFDI images of a moving mirror (e.g., amplitude: 0.78 mm, frequency: 30 Hz) are acquired at A-line rates of 8, 4, 2, and 1 kHz, respectively. The vertical axis represents the depth over 3.8 mm. The horizontal axis represents the time. The vibration amplitude in the images is artifactually increased as the A-line acquisition rate decreases (i.e., as the absolute sample movement during A-line acquisition increases).

For example, a moving sample can create a signal modulation even in the absence of tuning with the Doppler frequency: 2 V_{z}/λ, where V_{z} is the axial velocity and λ is the center optical wavelength. The Doppler frequency can be added to the original modulation frequency of the OFDI signal, resulting in an erroneous depth offset. The axial shift, z_{D} can be given by:

z_{image}=zₜᵣᵤₑz_{D};

z_{D}≈1.5(δz/λ)V_{Z}ΔT.

For example, δz is the axial resolution (e.g., about 10-15 μm) and ΔT is the A-line integration time (e.g., about 10-20 μs). Therefore, the Doppler axial shift (error) can be, e.g., 10-15 times of the actual displacement.

In a clinical setting, the vocal fold vibration can inevitably deviate from a perfect periodicity according to the patient's ability and the duration of the phonation. Exemplary procedures according to exemplary embodiments of the present disclosure can be implemented to simulate such non-ideal situations with the motorized stage and refine the exemplary procedures so that the variations in motion during image acquisition are detected and taken into account, as far as possible, during image reconstruction. An exemplary embodiment of a procedure according to the present disclosure can also be utilized to compensate for the Doppler-induced artifact based on the velocity map obtained from the OFDI images.

The fast 4D imaging capability can facilitate a quantitative analysis of various functional parameters of vocal folds. Clinically useful parameters can include a vibration amplitude map (in 3D and over time), a velocity map, a strain map, and an elasticity (Young's modulus) map.

To measure the vibration pattern, automatic image segmentation can be used to identify various anatomical structures in the vocal fold, such as the tissue surface, epithelial layer, and the junction between the epithelium and superficial lamina propria (SLP), as well as other heterogeneous features or injected materials. A motion tracking procedure can be applied to trace the movement of these microstructures in 3D over time from the sequence of reconstructed snapshot images. This exemplary analysis facilitate a reproduction of a vibration amplitude and velocity maps. Alternatively or in addition, the axial velocity of tissue motion can be directly measured by phase-sensitive OFDI procedure(s) and/or system(s) according to certain exemplary embodiments of the present disclosure.

An exemplary OCT-based elastography procedure for strain and elasticity mapping can be challenging because the short optical wavelengths used result in rapid noise- and strain-induced decorrelation of intensity patterns between consecutive image frames. In the past, motion tracking based on a frozen speckle assumption has not been successful for vascular optical elastography, particularly for structures on the size scale of arterial walls. (See Chan, R. C. et al. OCT-based arterial elastography: robust estimation exploiting tissue biomechanics, Optics Express 12, pp. 4558-4572 (2004). Therefore, it is possible to first minimize speckle by in- and out-of-plane frame averaging, taking advantage of the high-speed volumetric imaging capability of our system. This exemplary procedure can also facilitate a generation of the velocity map. A strain map can be calculated from the spatial derivative of the velocity map. Normally, the stress field that drives the vocal fold vibration is completely unknown. This can make it challenging to create a full tissue elasticity map, even with iterative numerical processing. To evaluate the initial feasibility of elastography, it is possible to investigate the relatively simple case of injected materials with a known viscoelasticity.

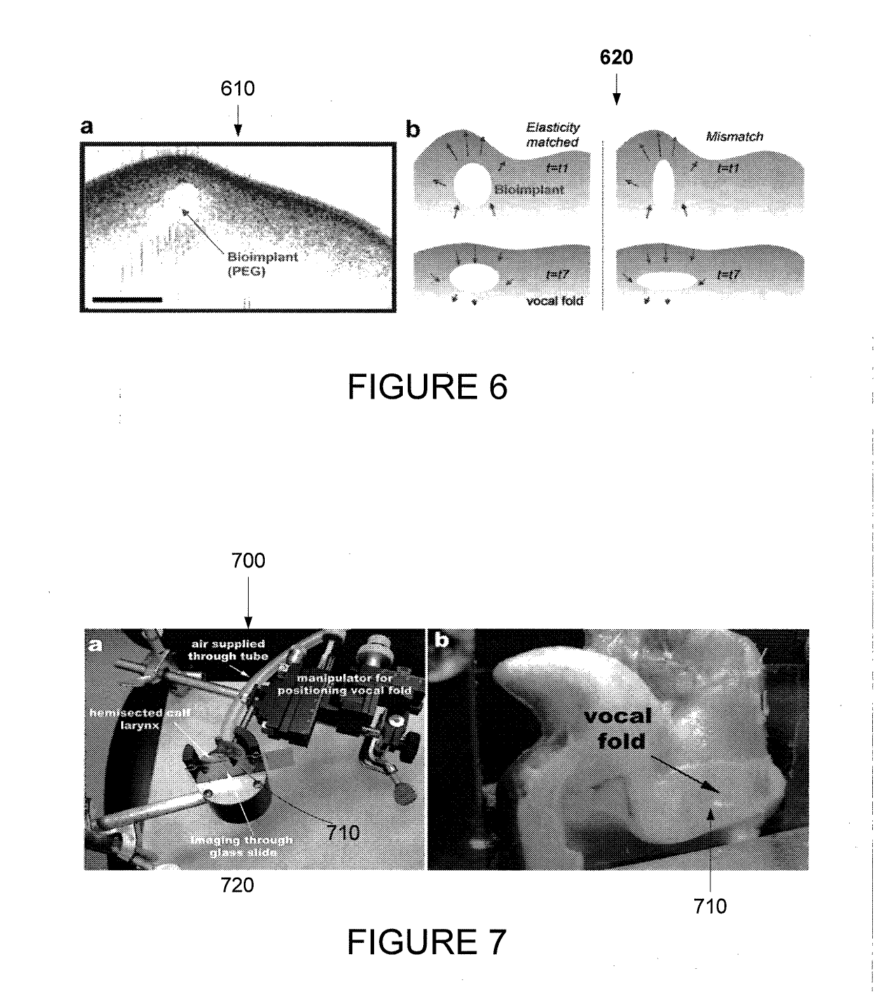

FIG. 6a shows an exemplary OFDI image 610 of the vocal fold after injecting PEG into the mucosa, so as to provide exemplary data and indicate an exemplary concept of elastography for characterizing biomechanical properties of implants in the vocal folds, in accordance with exemplary embodiments of the present disclosure. In particular, the exemplary OFDI image in FIG. 6a is that of a calf vocal fold ex vivo after injecting a polyethylene-glycol (PEG) based polymer gel, which is translucent so it shows up as white void. As the vocal fold is made to vibrate, the surrounding tissue undergoes elongation and compression and thus exerts alternating forces on the implant. For example, once the strain map and elastic modulus of the tissue are known, the exact stress field can be determined, and from the measured deformation of the bioimplant, its elastic modulus can be calculated. Further, it is possible to quantify the deformation of a number of materials, including PEG gel, saline and UV epoxy, with different Young's moduli and monitor the change in the deformability over time or in response to crosslinking in situ. FIG. 6b shows exemplary illustrations 620 of an expected deformation of the implant in the vibrating vocal fold so as to provide the exemplary data and indicate an exemplary concept of elastography for characterizing biomechanical properties of implants in the vocal folds, in accordance with exemplary embodiments of the present disclosure.

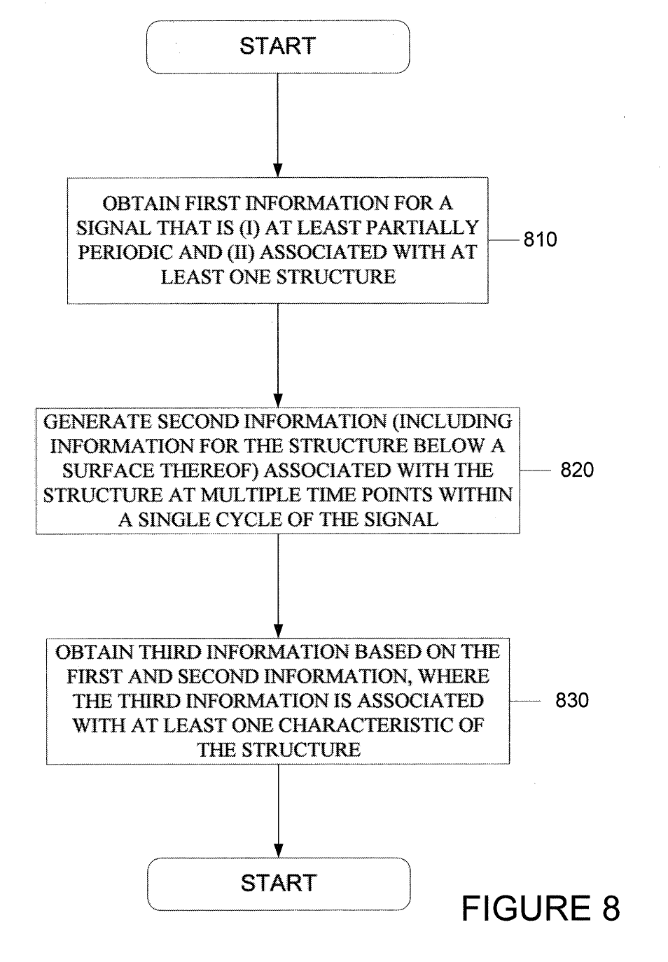

FIGS. 7a and 7b show exemplary images/photographs of exemplary vocal fold ex-vivo testing apparatus 700 according to an exemplary embodiments of the present disclosure, as well as an illustration of the vocal cord 710 which is analyzed thereby. For example, FIG. 7a illustrates the exemplary vocal fold ex-vivo testing apparatus 700 (which can be an exemplary OCT system) according to an exemplary embodiment of the present disclosure using which a hemisected larynx 710 is sealed in a chamber and warm humidified air is blown past the vocal fold, which is apposed to a glass slide. The vocal fold exhibits mucosal wave motion that can be similar to an intact larynx. The exemplary OCT system 700 can be positioned to view the medial surface of the vocal fold through the glass slide 720. A pressure transducer can be placed in the airway below the vocal folds and connected to a signal conditioner, amplifier and trigger circuit for synchronization. FIG. 7b illustrates an enlarged view of the bisected larynx 710 showing vocal fold against glass. In an alternate exemplary configuration according to the present disclosure, it is possible to utilize an intact larynx and view from directly above the vibrating vocal folds to better simulate human laryngoscopy.

Among certain preferred features of exemplary OFDI techniques and systems can be their compatibility with single-mode optical fiber delivery to the vocal fold through narrow diameter, flexible fiber-optic catheters. For example, a 2.8 mm (diameter) OCT catheter can be used for oral and laryngeal examination. The exemplary catheter can incorporate a micro-mirror scanner implemented with micro-electro-mechanical systems (MEMS) technology. Such exemplary catheter can be coupled to a spectral-domain OCT system for 3D endoscopic imaging of mucosa by direct contact to the tissue. This exemplary catheter can be used for 3D contact imaging of vocal folds in human patients undergoing laryngeal surgery, and to resolve vocal fold layers and details of vocal fold pathologies. Imaging vibrating vocal folds can use a non-contact long working distance optics, making the previous contact catheter design inadequate. According to the exemplary embodiments of the present disclosure, it is possible to determine optical design specifications, including the working distance and internal beam diameter, for the realization of rigid and eventually flexible transnasal catheters based on a MEMS scanner.

A reliable trigger signal can be obtained from an electroglottographic (EGG) waveform, a signal that tracks changes in electrical impedance across the vocal folds during their opening and closing. The EGG can be obtained using surface electrodes and an EGG instrument (e.g., Glottal Enterprises, EG-2). It is possible to use a system for synchronized capture of high-speed images and EGG signals. Using intact excised larynges, it is possible to optimize EGG-based triggering for OCT synchronization. Temporal landmarks in the glottal cycle can be extracted from the high-speed video using existing software for tracking the edges of the vocal folds across frames. The simultaneously acquired EGG signal can then be processed digitally to determine the filtering and triggering parameters (e.g., differentiation followed by Schmitt trigger) to minimize time jitter in the triggering. An analog trigger circuit for OCT synchronization can be provided based on those exemplary results.

Exemplary tradeoffs can exist between the time required to acquire a 3D data set and the spatio-temporal resolution of that data set. A short acquisition time has the advantage of being less susceptible to drift, while a long acquisition time could provide more detailed images if conditions are stable. It is possible to acquire data sets where we vary sampling density (number of cross-sectional planes or number of A-lines per plane), and then assess the results for how well they capture essential spatial and temporal features (e.g. the ability to clearly resolve the boundary between epithelium and SLP). The exemplary embodiments can utilize and/or have several modes of operation that are optimized for capturing different kinds of data. Such exemplary embodiments can assist in a definition of certain exemplary useful modes.

FIG. 8 shows a block diagram of a method according to an exemplary embodiment of the present disclosure. For example, in procedure 810, a first information can be obtained for at least one signal that is (i) at least partially periodic and (ii) associated with at least one structure. Then, at procedure 820, with a computer, a second information associated with the structure can be generated at multiple time points within a single cycle of the at least one signal. The second information can include information for the structure below a surface thereof. Further, at procedure 830, a third information can be provided that is based on the first information and the second information. The third information can be associated with at least one characteristic of the structure.

The foregoing merely illustrates the principles of the present disclosure. Various modifications and alterations to the described embodiments will be apparent to those skilled in the art in view of the teachings herein. For example, more than one of the described exemplary arrangements, radiations and/or systems can be implemented to implement the exemplary embodiments of the present disclosure Indeed, the arrangements, systems and methods according to the exemplary embodiments of the present invention can be used with and/or implement any OCT system, OFDI system, SD-OCT system or other imaging systems, and for example with those described in International Patent Application PCT/US2004/029148 filed Sep. 8, 2004 (which published as International Patent Publication No. WO 2005/047813 on May 26, 2005), U.S. patent application Ser. No. 11/266,779 filed Nov. 2, 2005 (which published as U.S. Patent Publication No. 2006/0093276 on May 4, 2006), U.S. patent application Ser. No. 10/861,179 filed Jun. 4, 2004, U.S. patent application Ser. No. 10/501,276 filed Jul. 9, 2004 (which published as U.S. Patent Publication No. 2005/0018201 on Jan. 27, 2005), U.S. patent application Ser. No. 11/445,990 filed Jun. 1, 2006, International Patent Application PCT/US2007/066017 filed Apr. 5, 2007, and U.S. patent application Ser. No. 11/502,330 filed Aug. 9, 2006, the disclosures of which are incorporated by reference herein in their entireties. It will thus be appreciated that those skilled in the art will be able to devise numerous systems, arrangements and methods which, although not explicitly shown or described herein, embody the principles of the present disclosure and are thus within the spirit and scope of the present disclosure. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. An apparatus comprising:
at least one first arrangement which is configured to obtain a first information for at least one signal that is (i) at least partially periodic and (ii) associated with at least one structure;
at least one second arrangement which is configured to generate a second information associated with the at least one structure at multiple time points within a single cycle of the at least one signal, wherein the second information includes information for the at least one structure below a surface thereof; and
at least one third arrangement which is configured to generate a third information based on the first information and the second information, wherein the third information is associated with at least one characteristic of the at least one structure.

2. The apparatus according to claim 1, wherein the first information includes first data for multiple time points within one cycle of the at least partially periodic signal.

3. The apparatus according to claim 1, wherein the third information includes at least one image associated with the at least one structure.

4. The apparatus according to claim 1, wherein the at least one image is a three-dimensional image.

5. The apparatus according to claim 3, wherein the at least one image includes multiple sequential images over the multiple time points.

6. The apparatus according to claim 1, wherein the third information includes velocity information of a periodic motion of the at least one structure during the multiple time points.

7. The apparatus according to claim 1, wherein the third information includes mechanical properties of the at least one structure during the multiple time points.

8. The apparatus according to claim 1, wherein the third information includes strain information for the at least one structure.

9. The apparatus according to claim 1, wherein the third information includes further information regarding a periodic motion of the at least one structure during the multiple time points.

10. The apparatus according to claim 1, wherein the at least one structure is at least one anatomical structure.

11. The apparatus according to claim 1, wherein the at least one structure includes polymers or viscoelastic materials.

12. The apparatus according to claim 1, wherein the at least one second arrangement includes an optical coherence tomography arrangement.

13. The apparatus according to claim 12, wherein the optical coherence arrangement is configured to transmit a radiation the at least one structure, and controls the radiation as a function the first information provided by the at least one first arrangement.

14. The apparatus according to claim 12, wherein the optical coherence arrangement is facilitated in an endoscope or a catheter.

15. The apparatus according to claim 12, wherein the second information includes a phase interference information associated with the at least one structure, and wherein the at least one third arrangement is configured to determine at least one characteristic of a motion of the at least one structure using the phase interference information.

16. The apparatus according to claim 12, wherein the at least one characteristic of the motion comprises an amplitude property of the motion.

17. The apparatus according to claim 16, wherein the radiation is controlled by controlling a propagation direction of the radiation.

18. The apparatus according to claim 1, wherein the at least one first arrangement obtains the first information during a motion of the at least structure.

19. The apparatus according to claim 18, wherein a periodicity of the motion is in a range of approximately 10 Hz and 10 KHz.

20. The apparatus according to claim 1, wherein the at least one structure is at least one vocal cord.

21. The apparatus according to claim 1, wherein the third information is provided for an internal portion of the at least one structure.

22. The apparatus according to claim 1, wherein the at least one first arrangement includes at least one of a piezoelectrical transducer, an ultrasound transducer, an optical position sensor, or an imaging arrangement which indicates a motion of or within the at least one structure.

23. A method comprising:
obtaining a first information for at least one signal that is (i) at least partially periodic and (ii) associated with at least one structure;
with a computer arrangement, generating a second information associated with the at least one structure at multiple time points within a single cycle of the at least one signal, wherein the second information includes information for the at least one structure below a surface thereof; and
providing a third information based on the first information and the second information, wherein the third information is associated with at least one characteristic of the at least one structure.
